(19) United States
(12) Patent Application Publication
(10) Pub. No.: US 2025/0120621 A1
(43) Pub. Date: Apr. 17, 2025

(54) PHYSIOLOGICAL MEASUREMENT SYSTEM WITH AUTOMATIC WAVELENGTH ADJUSTMENT

(71) Applicant: WILLOW LABORATORIES, INC., Irvine, CA (US)

(72) Inventors: Walter M. Weber, Laguna Hills, CA (US); Ammar Al-Ali, San Juan Capistrano, CA (US)

(21) Appl. No.: 18/894,772

(22) Filed: Sep. 24, 2024

Related U.S. Application Data
(63) Continuation of application No. 18/069,881, filed on Dec. 21, 2022, now Pat. No. 12,127,833.
Application No. 18/069,881 is a continuation of application No. 16/989,291, filed on Aug. 10, 2020, now Pat. No. 11,534,087.
Application No. 16/989,291 is a continuation of application No. 15/812,930, filed on Nov. 14, 2017, now Pat. No. 10,750,983.
Application No. 15/812,930 is a continuation of application No. 12/949,271, filed on Nov. 18, 2010, now Pat. No. 9,839,381.
(60) Provisional application No. 61/330,253, filed on Apr. 30, 2010.
(60) Provisional application No. 61/264,182, filed on Nov. 24, 2009.

(51) Int. Cl. A61B 5/1455; A61B 5/00; A61B 5/0205

(52) U.S. Cl. CPC A61B 5/14551; A61B 5/0059; A61B 5/0205; A61B 5/7203; A61B 5/7221; A61B 5/0075

(57) ABSTRACT

Disclosed herein is a physiological measurement system that can automatically adjust the number of wavelengths used based on the quality of a sensor signal that is reflective of an optical radiation detected at a sensor after tissue attenuation. The signal quality is examined to determine if it is sufficient to support the use of a full set of wavelengths. If it is determined to be insufficient to support the full set, a reduced number of wavelengths is used.

### PRIORITY CLAIM TO RELATED PROVISIONAL APPLICATIONS

The present application claims priority benefit under 35 U.S.C. § 119(e) to U.S. Provisional Patent Application Ser. No. 61/264,182, filed Nov. 24, 2009, entitled “PHYSIOLOGICAL MEASUREMENT SYSTEM WITH AUTOMATIC WAVELENGTH ADJUSTMENT,” and No. 61/330,253, filed Apr. 30, 2010, entitled “PHYSIOLOGICAL MEASUREMENT SYSTEM WITH AUTOMATIC WAVELENGTH ADJUSTMENT.” The present application incorporates the foregoing disclosures herein by reference.

### INCORPORATION BY REFERENCE OF COPENDING RELATED APPLICATIONS

The present application is related to the following copending U.S. utility applications:

| | App. Sr. No. | Filing Date | Title | Atty Dock. |
|---|---|---|---|---|
| 1 | 11/367,013 | Mar. 1, 2006 | Multiple Wavelength | MLR.002A |
| | | | Sensor Emitters | |
| 2 | 11/366,955 | Mar. 1, 2006 | Multiple Wavelength | MLR.003A |
| | | | Sensor Equalization | |
| 3 | 11/366,209 | Mar. 1, 2006 | Multiple Wavelength | MLR.004A |
| | | | Sensor Substrate | |
| 4 | 11/366,210 | Mar. 1, 2006 | Multiple Wavelength | MLR.005A |
| | | | Sensor Interconnect | |
| 5 | 11/366,833 | Mar. 1, 2006 | Multiple Wavelength | MLR.006A |
| | | | Sensor Attachment | |
| 6 | 11/366,997 | Mar. 1, 2006 | Multiple Wavelength | MLR.009A |
| | | | Sensor Drivers | |
| 7 | 11/367,034 | Mar. 1, 2006 | Physiological | MLR.010A |
| | | | Parameter | |
| | | | Confidence Measure | |
| 8 | 11/367,036 | Mar. 1, 2006 | Configurable | MLR.011A |
| | | | Physiological | |
| | | | Measurement System | |
| 9 | 11/367,033 | Mar. 1, 2006 | Noninvasive | MLR.012A |
| | | | Multi-Parameter | |
| | | | Patient Monitor | |
| 10 | 11/367,014 | Mar. 1, 2006 | Noninvasive | MLR.013A |
| | | | Multi-Parameter | |
| | | | Patient Monitor | |
| 11 | 11/366,208 | Mar. 1, 2006 | Noninvasive | MLR.014A |
| | | | Multi-Parameter | |
| | | | Patient Monitor | |
| 12 | 12/056,179 | Mar. 26, 2008 | Multiple Wavelength | MLR.015A |
| | | | Optical Sensor | |
| 13 | 12/082,810 | Apr. 14, 2008 | Optical Sensor | MLR.015A2 |
| | | | Assembly | |

The present application incorporates the foregoing disclosures herein by reference.

### BACKGROUND

Spectroscopy is a common technique for measuring the concentration of organic and some inorganic constituents of a solution. The theoretical basis of this technique is the Beer-Lambert law, which states that the concentration cᵢ of an absorbent in solution can be determined by the intensity of light transmitted through the solution, knowing the pathlength d_{λ}, the intensity of the incident light I_{0,λ}, and the extinction coefficient ε_{i,λ} at a particular wavelength λ. In generalized form, the Beer-Lambert law is expressed as:

$${I}_{λ} = I {\text{?}}_{, λ} {e}^{- {d}_{λ} \cdot μ {\text{?}}_{, λ}} \qquad ( 1 )$$

$$μ {\text{?}}_{, λ} = {\sum }_{i = 1}^{\text{?}} {ε}_{i , λ} \cdot {c}_{i} \qquad ( 2 )$$

$$\text{?} \text{indicates text missing or illegible when filed}$$

where μ_{α,λ} is the bulk absorption coefficient and represents the probability of absorption per unit length. The minimum number of discrete wavelengths that are required to solve EQS. 1-2 are the number of significant absorbers that are present in the solution.

A practical application of this technique is pulse oximetry, which utilizes a noninvasive sensor to measure oxygen saturation (SpO₂) and pulse rate. In general, the sensor has light emitting diodes (LEDs) that transmit optical radiation of red and infrared wavelengths into a tissue site and a detector that responds to the intensity of the optical radiation after absorption (e.g., by transmission or transreflectance) by pulsatile arterial blood flowing within the tissue site. Based on this response, a processor determines measurements for SpO₂, pulse rate, and can output representative plethysmographic waveforms. Thus, “pulse oximetry” as used herein encompasses its broad ordinary meaning known to one of skill in the art, which includes at least those noninvasive procedures for measuring parameters of circulating blood through spectroscopy. Moreover, “plethysmograph” as used herein (commonly referred to as “photoplethysmograph”), encompasses its broad ordinary meaning known to one of skill in the art, which includes at least data representative of a change in the absorption of particular wavelengths of light as a function of the changes in body tissue resulting from pulsing blood. Pulse oximeters capable of reading through motion induced noise are available from Masimo Corporation (“Masimo”) of Irvine, California. Moreover, portable and other oximeters capable of reading through motion induced noise are disclosed in at least U.S. Pat. Nos. 6,770,028, 6,658,276, 6,157,850, 6,002,952 5,769,785, and 5,758,644, which are owned by Masimo and are incorporated by reference herein. Such reading through motion oximeters have gained rapid acceptance in a wide variety of medical applications, including surgical wards, intensive care and neonatal units, general wards, home care, physical training, and virtually all types of monitoring scenarios.

FIG. 1 illustrates HbO₂ (Oxyhemoglobin) and Hb (Hemoglobin) absorption μ_{α} versus wavelength. At red and near IR wavelengths below 970 nm, where water has a significant peak, Hb and HbO₂ are the only significant absorbers normally present in the blood. Thus, typically only two wavelengths are needed to resolve the concentrations of Hb and HbO₂, e.g. a red (RD) wavelength at 660 nm and an infrared (IR) wavelength at 940 nm. In particular, SpO₂ is computed based upon a red ratio Red_{AC}/Red_{DC} and an IR ratio IR_{AC}/IR_{DC}, which are the AC detector response magnitude at a particular wavelength normalized by the DC detector response at that wavelength. The normalization by the DC detector response reduces measurement sensitivity to variations in tissue thickness, emitter intensity and detector sensitivity, for example. The AC detector response is a plethysmograph, as described above. Thus, the red and IR ratios can be denoted as NP_{RD} and NP_{IR} respectively, where NP stands for “normalized plethysmograph.” In pulse oximetry, oxygen saturation is calculated from the ratio NP_{RD}/NP_{IR}.

### SUMMARY OF THE DISCLOSURE

Embodiments of the disclosure are directed to a physiological measurement system that can automatically adjust the number of wavelengths used based on a sensor signal that is indicative of the optical radiation detected at the sensor after tissue attenuation. In an embodiment, the physiological measurement system performs a calibration process upon power up and/or a first attachment to a tissue site. During the calibration process, the system provides test currents to the light emitting sources in the emitter assembly and examines the sensor signal to determine if the signal quality is sufficient to support the use of a full set of wavelengths. The full set of wavelengths includes eight wavelengths in an embodiment. If it is determined that the signal quality is insufficient to support the full set, a reduced number of wavelengths is used. In an embodiment, the wavelengths at 660 nm and 905 nm, the minimum two wavelengths needed to provide a SpO2 reading, are used in lieu of the full set of wavelengths. In other embodiments, other reduced numbers of wavelengths are used. In other embodiments, the physiological measurement system continually monitors signal quality and automatically adjusts the number of wavelengths used.

### BRIEF DESCRIPTION OF THE DRAWINGS

FIG. 1 is a graph of oxyhemoglobin and reduced hemoglobin light absorption versus wavelength across portions of the red and IR spectrum;

FIG. 2 is a graph of NP ratios versus wavelength illustrating a tissue profile;

FIG. 3A is a perspective view of a physiological measurement system utilizing a multiple wavelength sensor;

FIG. 3B is a perspective view of a multiple wavelength sensor embodiment;

FIG. 4A is a general block diagram of a multiple wavelength sensor and sensor controller;

FIG. 4B is a general block diagram of a monitor and a sensor;

FIG. 4C is a general block diagram of an emitter assembly;

FIG. 5A is a general block diagram of an emitter array;

FIG. 5B is a schematic diagram of an emitter array embodiment;

FIGS. 6A-6C are flow diagrams illustrating automatic wavelength adjustment processes in accordance with various embodiments;

FIG. 7A is a graph of NP ratios versus wavelength illustrating a probe-off profile;

FIG. 7B is a graph of NP ratios versus wavelength illustrating a penumbra profile;

FIG. 8 is a general block diagram of a confidence measurement system;

FIG. 9A is a graph of normalized plethysmograph (NP) ratios versus wavelength for low and high SpO₂ illustrating a NP envelope;

FIG. 9B is a block diagram of a multiple wavelength probe off detector utilizing an NP envelope;

FIG. 10A is a graph of NP ratios versus wavelength illustrating a family of parametric NP curves;

FIG. 10B is a block diagram of a multiple wavelength confidence measurement system utilizing parametric NP curves;

FIG. 11A is an NP ratio graph illustrating a family of NP data clusters;

FIG. 11B is a block diagram of a multiple wavelength confidence measurement system utilizing NP data clusters; and

FIG. 12 is a graph showing a ratio of normalized detector signal to current provided to an LED.

### DETAILED DESCRIPTION OF THE PREFERRED EMBODIMENTS

In this application, reference is made to many blood parameters. Some references that have common shorthand designations are referenced through such shorthand designations. For example, as used herein, HbCO designates carboxyhemoglobin, HbMet designates methemoglobin, and Hbt designates total hemoglobin. Other shorthand designations such as COHb, MetHb, and tHb are also common in the art for these same constituents. These constituents are generally reported in terms of a percentage, often referred to as saturation, relative concentration or fractional saturation. Total hemoglobin is generally reported as a concentration in g/dL. The use of the particular shorthand designators presented in this application does not restrict the term to any particular manner in which the designated constituent is reported.

Embodiments of the disclosure are directed to a physiological measurement system that can automatically adjust the number of wavelengths used based on a sensor signal that is indicative of the optical radiation detected at the sensor after tissue attenuation. In various embodiments, the adjustment process utilizes various methods of NP profile comparison to derive a confidence measurement to measure the quality of the signal detected at the sensor. In other embodiments, the system provides test currents to light emitter sources in the emitter assembly and measure sensor signals in response to the light emitted to determine signal quality. If the signal quality is insufficient to support using the full set of wavelengths, the physiological measurement system can switch to using less than the full set of wavelengths.

### Example Normalized Plethysmograph (NP) Tissue Profile

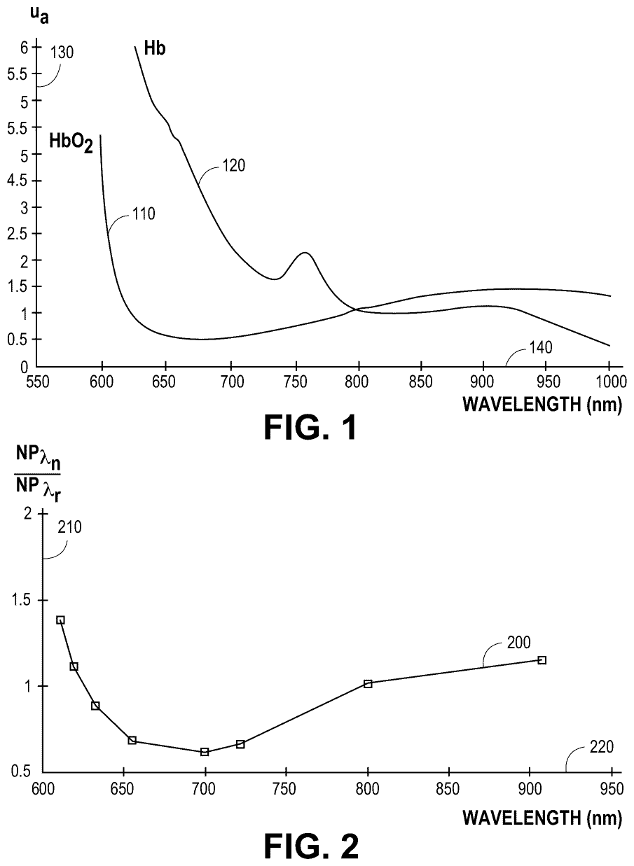

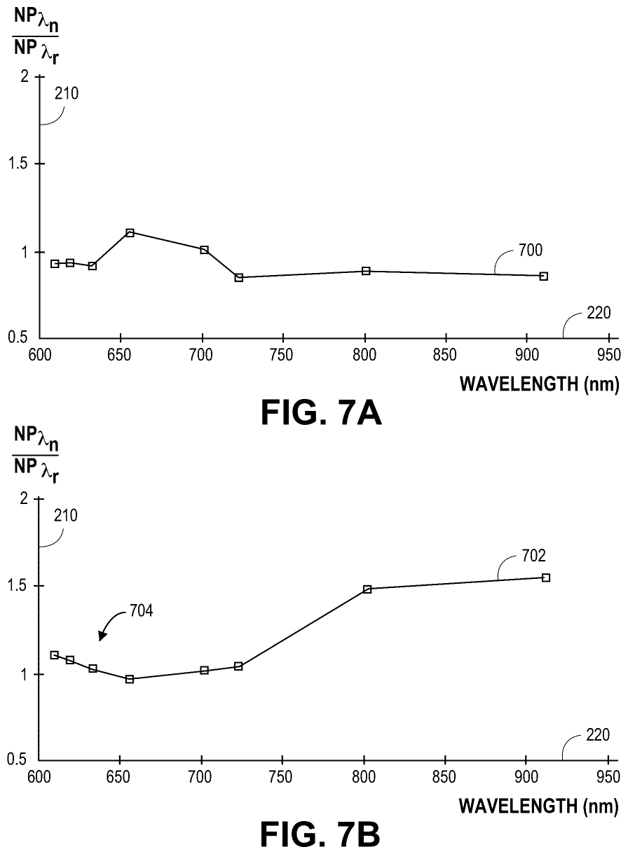

FIG. 2 illustrates an example of a “tissue profile” 200 for SpO2=97%. For this example, including FIGS. 7A-7B, below, the sensor emits eight wavelengths (610, 620, 630, 655, 700, 720, 800 and 905 nm). The graph is a plot of NP ratios 210 versus wavelength 220, where the NP ratios are of the form NP_{λ1}/NP_{λ2}. This is a generalization to multiple wavelengths of the ratio NP_{RD}/NP_{IR} described above in FIG. 1 for two (red and IR) wavelengths. In order to provide a common scale for these NP ratios, the ratios are calculated with respect to a reference wavelength, λr, which may be any of the available wavelengths. Thus, the plotted NP ratios are denoted NP_{λn}/NP_{λr} over the n available wavelengths, including λr. Note that the NP ratio at the reference wavelength is NP_{λr}/NP_{λr}=1, which is 800 nm in FIG. 2.

As shown in FIG. 2, when a sensor is properly positioned on a tissue site, the detector only receives LED emitted light that has propagated through the tissue site after tissue scattering and absorption. Thus, a tissue profile 200 should reflect the blood constituent absorption characteristics illustrated in FIG. 1, above. For this high oxygen saturation (97%) example, HbO₂ is the only significantly absorbing blood constituent and, indeed, the resulting tissue profile 200 is shaped like the HbO₂ absorption curve 110 (FIG. 1).

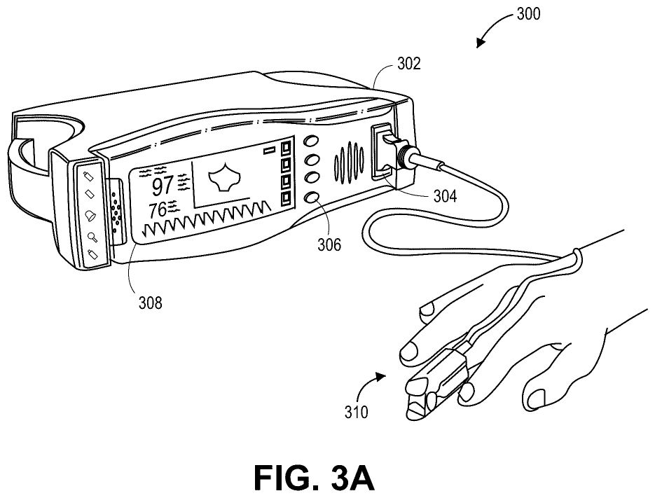

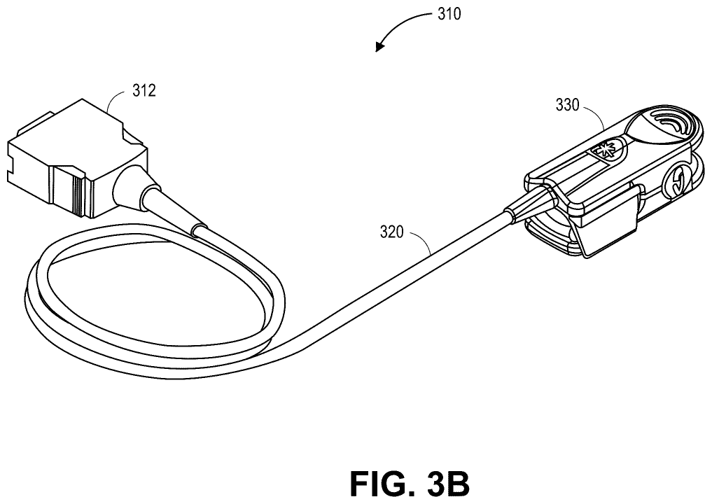

FIG. 3A illustrates an example physiological measurement system 300 that can output and detect wavelength profiles similar to that shown in FIG. 2. In an embodiment, the measurement system 300 includes a monitor 302 and a multiple wavelength sensor assembly 310 with enhanced measurement capabilities as compared with conventional pulse oximetry. The physiological measurement system 300 allows the monitoring of a person, including a patient. In particular, the multiple wavelength sensor assembly 310 allows the measurement of blood constituent and related parameters in addition to oxygen saturation and pulse rate. Alternatively, the multiple wavelength sensor assembly 310 allows the measurement of oxygen saturation and pulse rate with increased accuracy or robustness as compared with conventional pulse oximetry.

In an embodiment, the sensor assembly 310 is configured to plug into a monitor sensor port 304. Monitor keys 306 provide control over operating modes and alarms, to name a few. A display 308 provides readouts of measured parameters, such as oxygen saturation, pulse rate, HbCO and HbMet to name a few.

FIGS. 3B illustrates a multiple wavelength sensor assembly 310 having a sensor 330 adapted to attach to a tissue site, a sensor cable 320 and a monitor connector 312. In an embodiment, the sensor 330 is incorporated into a reusable finger clip adapted to removably attach to, and transmit light through, a fingertip. The sensor cable 320 and monitor connector 312 are integral to the sensor 330, as shown. In alternative embodiments, the sensor 330 may be configured separately from the cable 320 and connector 312.

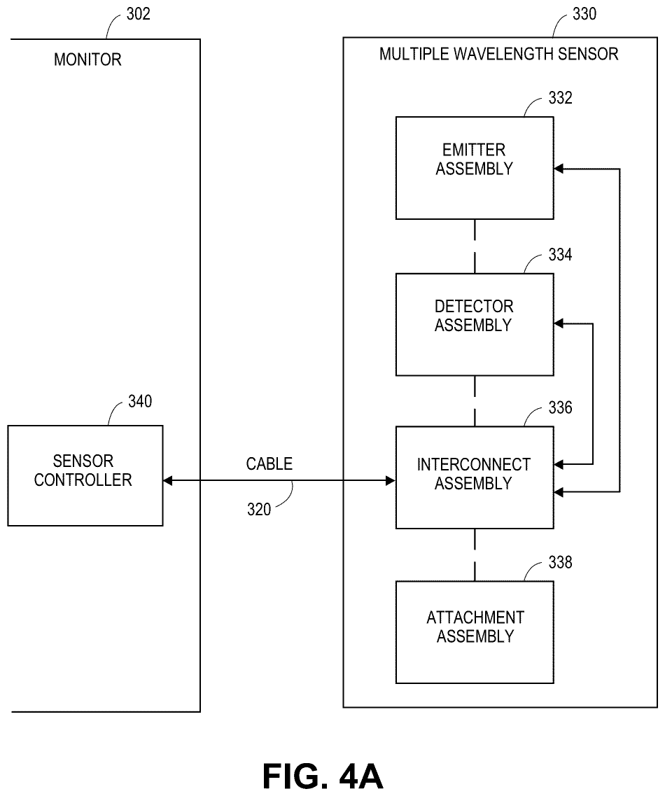

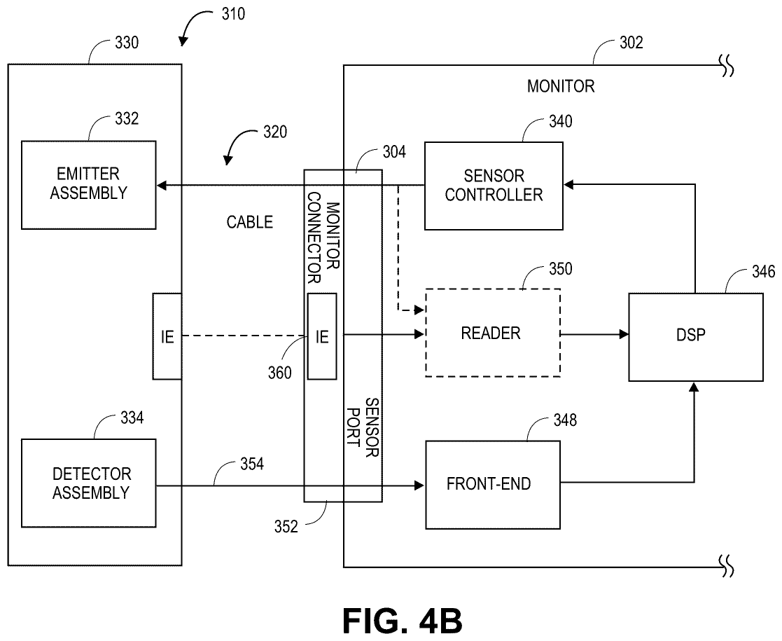

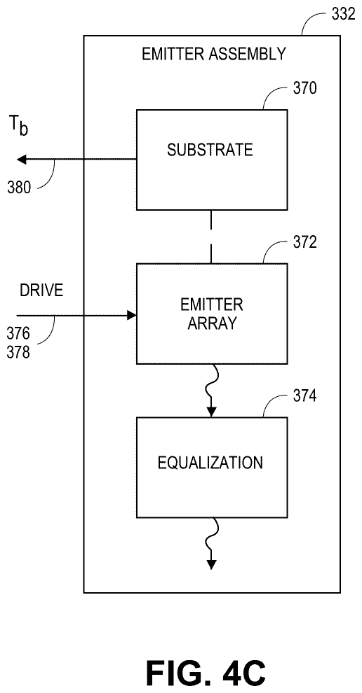

FIG. 4A illustrates the sensor 330 having an emitter assembly 332, a detector assembly 334, an interconnect assembly 336 and an attachment assembly 338. The emitter assembly 332 responds to drive signals received from a sensor controller 340 in the monitor 302 via the cable 320 so as to transmit optical radiation having a plurality of wavelengths into a tissue site. The detector assembly 334 provides a sensor signal to the monitor 302 via the cable 320 in response to optical radiation received after attenuation by the tissue site. The interconnect assembly 336 provides electrical communication between the cable 320 and both the emitter assembly 332 and the detector assembly 334. The attachment assembly 338 attaches the emitter assembly 332 and detector assembly 334 to a tissue site, as described above. Additional details of the detector assembly 334, the interconnect assembly 336 and the attachment assembly 338 are further described in the above-referenced application Ser. No. 11/367,013, filed Mar. 1, 2006, entitled “Multiple Wavelength Sensor Emitters,” which has been incorporated by reference above. The emitter assembly 332 will be described in further details below.

FIG. 4B illustrates a monitor 302 and a corresponding sensor assembly 310, as described generally with respect to FIGS. 3A, 3B and 4A above. As discussed above, the sensor assembly 310 houses the emitter assembly 332 having emitters. In an embodiment, the emitter assembly 332 is responsive to drivers within a sensor controller 340 so as to transmit optical radiation into a tissue site. The sensor 330 also houses a detector assembly 334 that provides a sensor signal 354 responsive to the optical radiation after tissue attenuation. In an embodiment, the sensor signal 354 is filtered, amplified, sampled and digitized by a front-end 348 and input to a DSP (digital signal processor) 346, which also commands the sensor controller 340. The sensor cable 320 electrically communicates drive signals from the sensor controller 340 to the emitter assembly 332 and a sensor signal 354 from the detector assembly 334 to a front-end 348. The sensor cable 320 has a monitor connector 352 that plugs into a monitor sensor port 304.

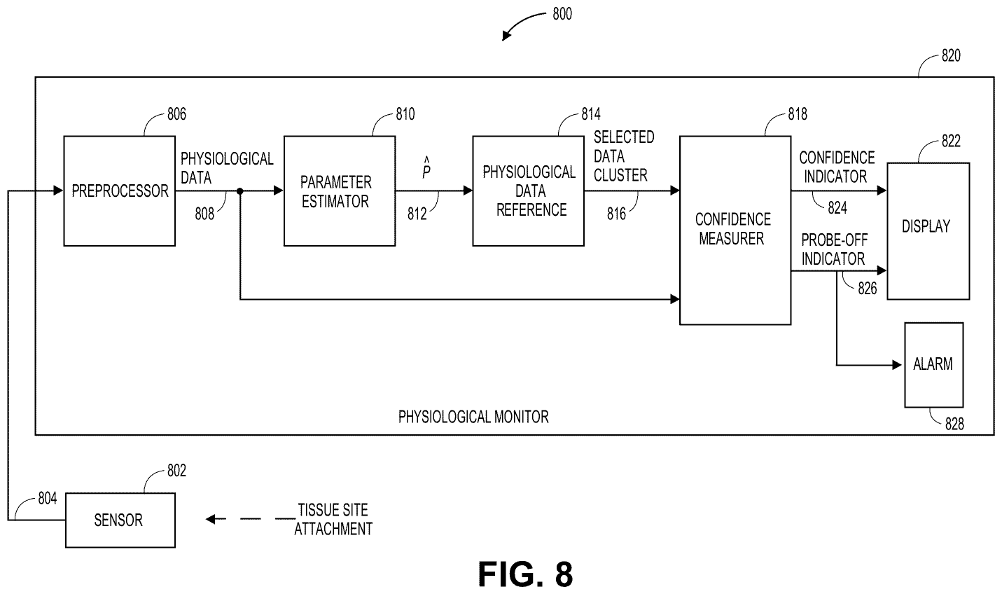

In an embodiment, the DSP 346 processes the incoming digitalized sensor signal 354 and determines whether the signal quality requires a change to the number of wavelengths that are active in the emitter assembly. In an embodiment, the DSP 346 includes methods and components for determining signal quality as shown in FIGS. 8A-12, as will be further described below.

In an embodiment, the monitor 302 also has a reader 350 capable of obtaining information from an information element (IE) 360 in the sensor assembly and transferring that information to the DSP 346, to another processor or component within the monitor 302, or to an external component or device that is at least temporarily in communication with the monitor 302. In an alternative embodiment, the reader function is incorporated within the DSP 346, utilizing one or more of DSP I/O, ADC, DAC features and corresponding processing routines, as examples. Additional details and alternate embodiments for components shown in FIG. 4B are further described in FIGS. 41-46 of the above-referenced application Ser. No. 11/367,013, filed Mar. 1, 2006, entitled “Multiple Wavelength Sensor Emitters.”

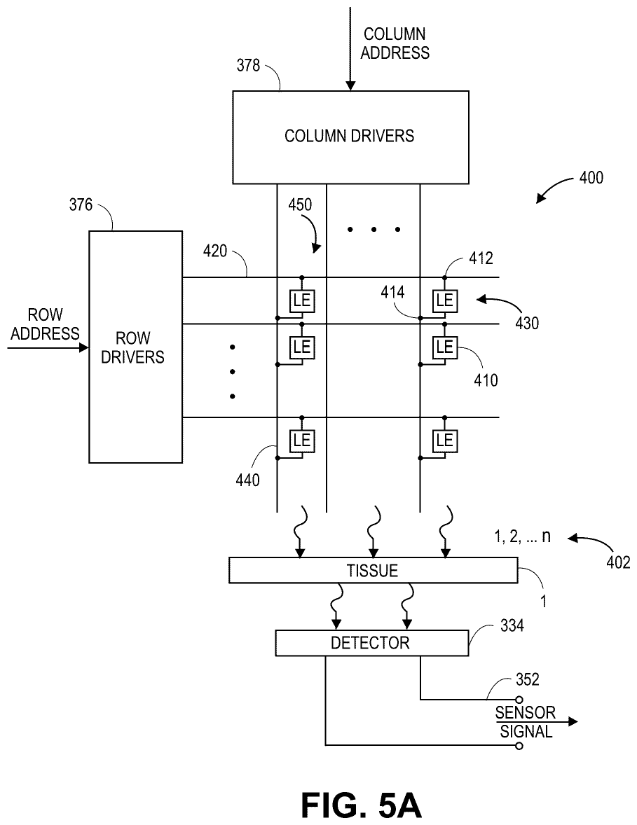

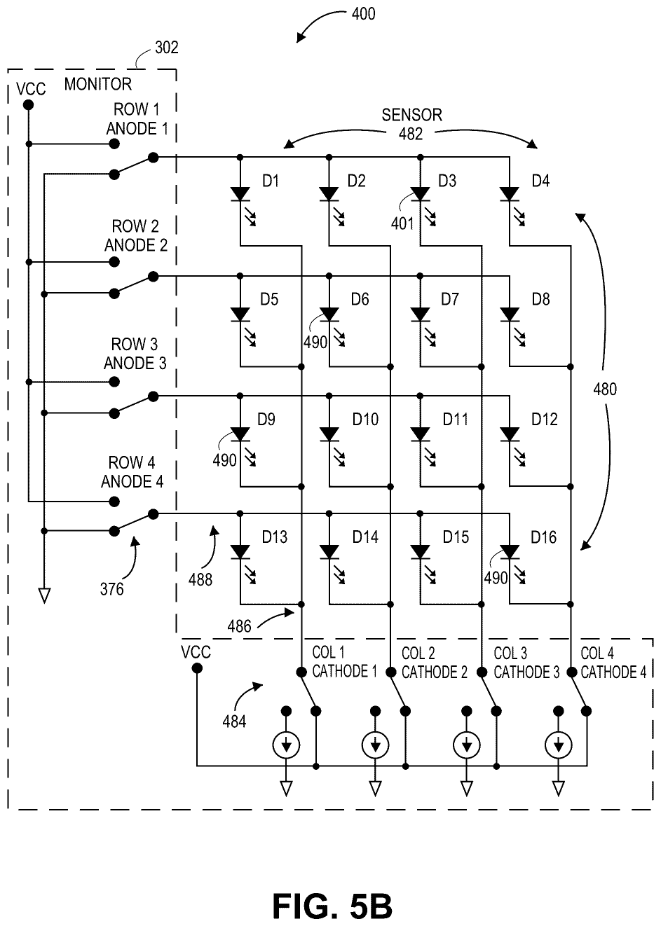

In an embodiment, the monitor connector 352 houses the information element 360, which may be a memory device or other active or passive electrical component. In a particular embodiment, the information element 360 is an EPROM, or other programmable memory, or an EEPROM, or other reprogrammable memory, or both. In an alternative embodiment, the information element 360 is housed within the sensor 330, or an information element 360 is housed within both the monitor connector 352 and the sensor 330. In yet another embodiment, the emitter assembly 332 has an information element 360, which is read in response to one or more drive signals from the sensor controller 340. In a further embodiment, a memory information element is incorporated into the emitter array 400 (FIG. 5A) and has characterization information relating to the LEDs 490 (FIG. 5B). In one advantageous embodiment, trend data relating to slowly varying parameters, such as perfusion index, HbCO or METHb, to name a few, are stored in an IE memory device, such as EEPROM.

### Emitter Assembly

FIG. 4C illustrates an emitter assembly 332 having an emitter array 372, a substrate 370 and equalization 374. The emitter array 372 has multiple light emitting sources, each activated by addressing at least one row and at least one column of an electrical grid. The light emitting sources are capable of transmitting optical radiation having multiple wavelengths. The equalization 374 accounts for differences in tissue attenuation of the optical radiation across the multiple wavelengths so as to at least reduce wavelength-dependent variations in detected intensity. The substrate 370 provides a physical mount for the emitter array and emitter-related equalization and a connection between the emitter array and the interconnection assembly. Advantageously, the substrate 370 also provides a bulk temperature measurement so as to calculate the operating wavelengths for the light emitting sources. The equalization 374 and the substrate 370 are described in further detail in above-referenced application Ser. No. 11/367,013, filed Mar. 1, 2006, entitled “Multiple Wavelength Sensor Emitters,” which has been incorporated by reference above.

### Emitter Array

FIG. 5A illustrates an emitter array 400 having multiple light emitters (LE) 410 capable of emitting light 402 having multiple wavelengths into a tissue site 1. The emitter array 400 emits optical radiation having multiple wavelengths of predetermined nominal values, advantageously allowing multiple parameter measurements. In particular, the emitter array 400 has multiple light emitting diodes (LEDs) 410 that are physically arranged and electrically connected in an electrical grid to facilitate drive control, equalization, and minimization of optical pathlength differences at particular wavelengths. In an embodiment, an optical filter is advantageously configured to provide intensity equalization across a specific LED subset. The substrate 370 is configured to provide a bulk temperature of the emitter array 400 so as to better determine LED operating wavelengths.

As shown in FIG. 5A, row drivers 376 and column drivers 378 are electrically connected to the light emitters 410 and activate one or more light emitters 410 by addressing at least one row 420 and at least one column 440 of an electrical grid. In an embodiment, the light emitters 410 each include a first contact 412 and a second contact 414. The first contact 412 of a first subset 430 of light emitters is in communication with a first conductor 420 of the electrical grid. The second contact 414 of a second subset 450 of light emitters is in communication with a second conductor 440. In an embodiment, each subset comprises at least two light emitters, and at least one of the light emitters of the first and second subsets 430, 450 are not in common. A detector 334 is capable of detecting the emitted light 402 and outputting a sensor signal responsive to the emitted light 402 after attenuation by the tissue site 1 via monitor connector 352. As such, the sensor signal is indicative of at least one physiological parameter corresponding to the tissue site 1, as described above.

FIG. 5B illustrates an emitter array 400 having LEDs 490 connected within an electrical grid of n rows and m columns totaling n+m drive lines 488, 486, where n and m integers greater than one. The electrical grid advantageously minimizes the number of drive lines required to activate the LEDs 490 while preserving flexibility to selectively activate individual LEDs 490 in any sequence and multiple LEDs 490 simultaneously. The electrical grid also facilitates setting LED currents so as to control intensity at each wavelength, determining operating wavelengths and monitoring total grid current so as to limit power dissipation. The emitter array 400 is also physically configured in rows 480. This physical organization facilitates clustering LEDs 490 according to wavelength so as to minimize pathlength variations and facilitates equalization of LED intensities.

As shown in FIG. 5B, one embodiment of an emitter array 400 comprises up to sixteen LEDs 490 configured in an electrical grid of four rows 480 and four columns 482. Each of the four row drive lines 488 provide a common anode connection to four LEDs 490, and each of the four column drive lines 486 provide a common cathode connection to four LEDs 490. Thus, the sixteen LEDs 490 are advantageously driven with only eight wires, including the four anode drive lines and the four cathode drive lines as shown. This compares favorably to conventional common anode or cathode LED configurations, which require more drive lines. In a particular embodiment, the emitter array 400 is partially populated with eight LEDs having nominal wavelengths as shown in TABLE 1. Further, LEDs having wavelengths in the range of 610-630 nm are grouped together in the same row. The emitter array 400 is adapted to a physiological measurement system 300 (FIG. 3A) for measuring H_{b}CO and/or METHb in addition to SₚO₂ and pulse rate.

TABLE 1
Nominal LED Wavelengths

| LED | λ | Row | Col |
|---|---|---|---|
| D1 | 630 | 1 | 1 |
| D2 | 620 | 1 | 2 |
| D3 | 610 | 1 | 3 |
| D4 | | 1 | 4 |
| D5 | 700 | 2 | 1 |
| D6 | 730 | 2 | 2 |
| D7 | 660 | 2 | 3 |
| D8 | 805 | 2 | 4 |
| D9 | | 3 | 1 |
| D10 | | 3 | 2 |
| D11 | | 3 | 3 |
| D12 | 905 | 3 | 4 |
| D13 | | 4 | 1 |
| D14 | | 4 | 2 |
| D15 | | 4 | 3 |
| D16 | | 4 | 4 |

Also shown in FIG. 5B, row drivers 376 and column drivers 484 located in the monitor 302 selectively activate the LEDs 490. In particular, row and column drivers 376, 484 function together as switches to Vcc and current sinks, respectively, to activate LEDs and as switches to ground and Vcc, respectively, to deactivate LEDs. This push-pull drive configuration advantageously prevents parasitic current flow in deactivated LEDs. In a particular embodiment, only one row drive line 488 is switched to Vcc at a time. One to four column drive lines 486, however, can be simultaneously switched to a current sink so as to simultaneously activate multiple LEDs within a particular row. LED drivers and the process of facilitating intensity equalization through the activation of two or more LEDs of the same wavelength are further described in the above-referenced application Ser. No. 11/367,013, filed Mar. 1, 2006, entitled “Multiple Wavelength Sensor Emitters.”

Although an emitter assembly is described above with respect to an array of light emitters each configured to transmit optical radiation centered around a nominal wavelength, in another embodiment, an emitter assembly advantageously utilizes one or more tunable broadband light sources, including the use of filters to select the wavelength, so as to minimize wavelength-dependent pathlength differences from emitter to detector. In yet another emitter assembly embodiment, optical radiation from multiple emitters each configured to transmit optical radiation centered around a nominal wavelength is funneled to a tissue site point so as to minimize wavelength-dependent pathlength differences. This funneling may be accomplished with fiberoptics or mirrors, for example. In further embodiments, the LEDs 490 can be configured with alternative orientations with correspondingly different drivers among various other configurations of LEDs, drivers and interconnecting conductors.

### Automatic Wavelength Adjustment Processes

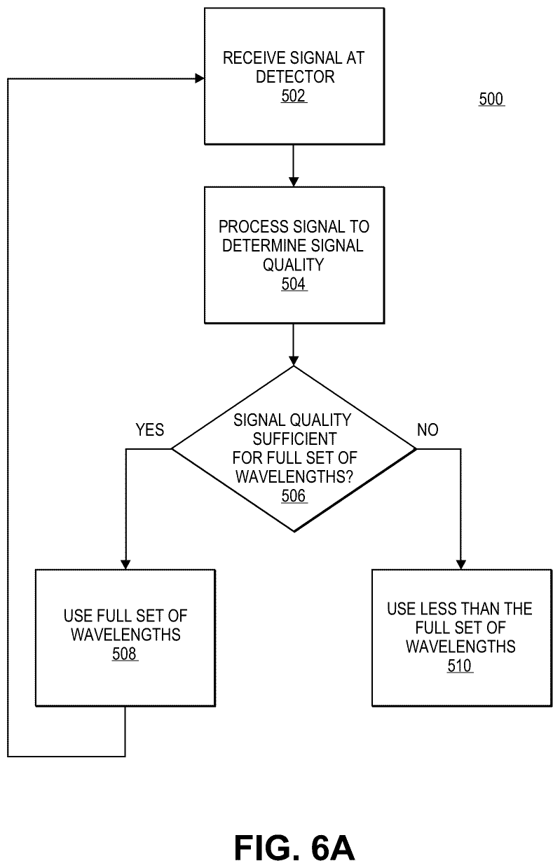

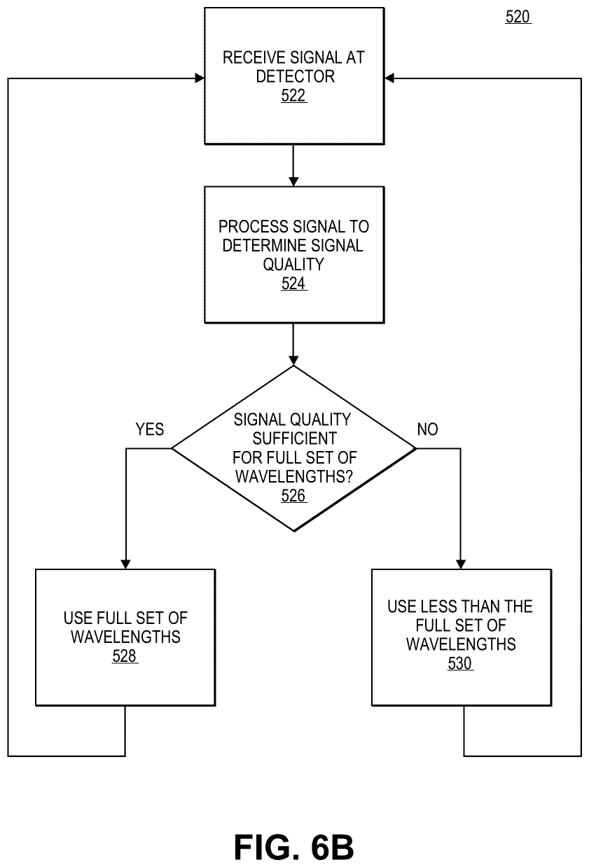

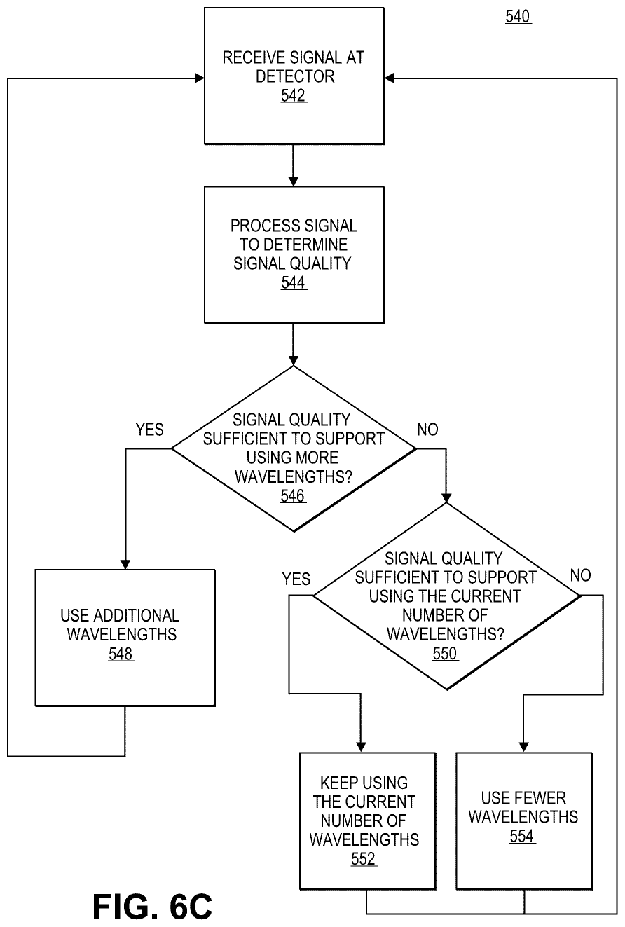

FIGS. 6A-6C are flow diagrams that illustrate the automatic wavelength adjustment processes in accordance with various embodiments. FIG. 6A illustrates an automatic wavelength adjustment process 500. In an embodiment, the process 500 is executed as part of or during a calibration process that is executed when the physiological measurement system 300 is first powered up and/or when the sensor assembly 310 is attached or re-attached to a tissue site. In another embodiment, the process 500 is executed periodically when the physiological measurement system 300 is in use.

As shown, the process 500 begins in an embodiment at block 502 with the detector 334 receiving a signal after tissue attenuation as described with respect to FIG. 4A. At block 504, the received signal is processed to determine signal quality. In an embodiment, the DSP 346 is configured to process the received signal that has been digitalized by the front-end 348 to determine signal quality. At block 506, the signal quality is evaluated to determine if it is sufficient to support a full set of active wavelengths. In an embodiment, the full set of active wavelengths includes the eight wavelengths as set forth in TABLE 1 above.

If the signal quality is determined to be lower than that which is needed to support the full set of active wavelengths, at block 510 the physiological measurement system 300 will use less than the full set of active wavelengths. In an embodiment, the DSP 346 sends a signal to the sensor controller 340 (FIG. 4B) to effectuate the use of less than the full set of active wavelengths. In an embodiment, the two active wavelengths used are at 660 nm (Red) and 905 nm (IR), the minimum two needed to detect SpO2. With reference to TABLE 1 and FIG. 5B, for example, LEDs D7 and D12 would be activated at block 510 while the rest of LEDs remain inactive.

In the alternative, if the signal quality is deemed to be sufficient to support the full set of active wavelengths, then at block 508 the physiological measurement system 300 will use the full set of active wavelengths. In an embodiment, the full set of active wavelengths includes the eight shown in TABLE 1. For example, the corresponding LEDs shown in TABLE 1 would be activated at block 508. In an embodiment, the process 500 then begins again at block 502. Various methods of determining and evaluating signal quality, including criteria for determining sufficiency of a signal quality to support a full set of active wavelengths, will be further described with respect to FIGS. 8A-12.

FIG. 6B shows another process 520 for automatic wavelength adjustment in which the physiological measurement system 300 periodically determines whether the full set of wavelengths should be used. The process 520 begins in an embodiment at block 522 with the detector 334 receiving a signal after tissue attenuation as described with respect to FIG. 4A. At block 524, the received signal is processed to determine signal quality. In an embodiment, the DSP 346 is configured to process the received signal that has been digitalized by the front-end 348 to determine signal quality. At block 526, the signal quality is evaluated to determine whether it is sufficient to support a full set of active wavelengths. In an embodiment, the full set of active wavelengths includes the eight wavelengths as set forth in TABLE 1 above.

If the signal quality is deemed to be lower than that which is needed to support the full set of active wavelengths, then at block 530 the physiological measurement system 300 will use less than the full set of active wavelengths. The DSP 346 can send a change signal to the sensor controller 340 (FIG. 4B) if the physiological measurement system 300 is currently using the full set of active wavelengths. For example, the change may reduce the number of active wavelengths from the eight shown in TABLE 1 to two (e.g., 660 nm (Red) and 905 nm (IR)). However, if the physiological measurement system 300 is already using less than the full set of active wavelengths, no action is performed at block 530. In either case, the process 520 returns to block 522 where a new signal will be received and processed at the next sampling cycle.

In the alternative, if the signal quality is deemed to be sufficient to support the full set of active wavelengths, then at block 528 the physiological measurement system 300 will either continue using the full set of active wavelengths (if the full set is already used) or change to using the full set of active wavelengths (if less than the full set is being used). If a change is needed, in an embodiment the DSP 346 can send a change signal to the sensor controller 340. The process 520 then returns to block 522, where a new signal will be received and processed at the next sampling cycle.

FIG. 6C shows another process 540 for automatic wavelength adjustment in which the physiological measurement system 300 periodically adjusts the number of wavelengths used depending on the detected signal quality. The process 540 begins in an embodiment at block 542 with the detector 334 receiving a signal after tissue attenuation as described with respect to FIG. 4A. At block 544, the received signal is processed to determine signal quality. In an embodiment, the DSP 346 is configured to process the received signal that has been digitalized by the front-end 348 to determine signal quality. At block 546, the signal quality is evaluated to determine whether it is sufficient to support additional active wavelengths. If so, then at block 548 the physiological measurement system 300 will use additional active wavelengths (if less than the full set is being used). If a change is needed, in an embodiment the DSP 346 can send a change signal to the sensor controller 340. The process 540 then returns to block 542, where a new signal will be received and processed at the next sampling cycle.

If the signal quality is deemed to not be sufficient to support more active wavelengths than those that are currently being used, then at block 550 the physiological measurement system 300 will determine whether the signal qualify can at least support the current set of active wavelengths. If it is sufficient, no action is taken at block 552 and the current number of active wavelengths will continue to be used. Otherwise, the physiological measurement system 300 will use fewer wavelengths. The DSP 346 can send a change signal to the sensor controller 340 (FIG. 4B). In any case, the process 540 returns to block 542 where a new signal will be received and processed at the next sampling cycle.

In various embodiments, portions of processes described in FIGS. 6A-6C can be performed at the front-end 348, the sensor controller 340, the DSP 346 or any other component within physiological measurement system 300.

### Signal Quality Determination

FIGS. 7A-7B illustrate profiles of two conditions that are indicative of degraded signal quality. FIGS. 8A-11B describe example methods of deriving a confidence measurement that can be used to measure signal quality, and in particular, to detect degraded signal quality shown in the examples illustrated below.

FIG. 7A illustrates an example of a probe-off profile 700. When a sensor is completely dislodged from a patient, a so-called “probe off” condition occurs. Despite a probe off condition, an optical sensor may continue to detect an AC signal, which can be induced at the detector by other than pulsatile arterial absorption of LED emitted light. For example, small patient movements, vibrations, air flow or other perturbations may cause the pathlength between the LEDs and the detector to vary, resulting in an AC detector signal that can be mistakenly interpreted by the monitor as due to pulsatile arterial blood. Further, ambient light may reach the detector, and any modulation of the ambient light due to AC power, power fluctuations, moving objects, such as a fan, among other perturbations can be also mistaken as a pulsatile arterial signal. Probe off errors are serious because a blood constituent monitor may display normal results, such as oxygen saturation, when, in fact, the sensor is not properly attached to the patient, potentially leading to missed severe desaturation events. As shown in FIG. 7A, a probe-off profile 700 is readily apparent as it does not have a shape related to the absorption characteristics of hemoglobin constituents.

FIG. 7B illustrates an example of a penumbra profile 702. When a sensor is not properly positioned or becomes partially dislodged, a penumbra condition may occur, where the detector is “shadowed” by a tissue site, such as a finger, but also receives some light directly from the emitters or indirectly reflected off the sensor housing, or both. As a result, the DC signal at the detector rises significantly, which lowers the AC/DC ratio (NP). Because red wavelengths are more significantly absorbed by Hb and HbO2, the penumbra condition is most noticeable at the red portion 704 of the NP_{λn}/NP_{λr}. This effect is readily seen in the penumbra profile 702 as compared to a normal tissue profile 200 (FIG. 2).

Advantageously, a physiological parameter confidence measurement system, as described below, can distinguish a tissue profile 200 (FIG. 2) from a probe-off profile 700 (FIG. 7A) or penumbra profile 702 (FIG. 7B), as examples. Further, a physiological parameter confidence measurement system can provide indications that the detector signal is degraded as the result of various physiological and non-physiological phenomenons.

### Physiological Parameter Confidence Measurement System

FIG. 8 illustrates a physiological parameter confidence measurement system 800 having a physiological data 808 input, a confidence indicator 824 output and a probe-off indicator 826 output. In an embodiment, physiological data 808, such as the NP ratios described above, is derived from a sensor 802 generating a sensor signal 804 responsive to multiple wavelengths of optical radiation transmitted into and attenuated by a tissue site. The confidence indicator 824 provides an observer with some measure of “goodness” for the physiological data 808. That is, if confidence is high, it is likely the physiological data 816 is representative of a physiological condition or state. If confidence is low, the physiological data 808 may be less representative of a physiological condition or state. If the confidence is very low, a probe-off indicator 826 may be generated to alert an observer to the possibility that a sensor from which the physiological data 808 is derived is not properly positioned on a tissue site and may not be generating physiologically significant data. In an embodiment, a confidence measure may be provided as a percentage, such as 0-100%.

The confidence measure can be used to measure signal quality in the processes described above with respect to FIGS. 6A-6C. For example, the confidence level threshold may be set at 80% in order for a full set of wavelengths to be used. In other embodiments, the threshold may be set by the user of the physiological measurement system 300. In various embodiments, a confidence indicator 824 corresponding to a confidence measure may be visual (through a display 822) or audible (through an alarm 828) or both. The visual or audible indication may assist the user in setting the threshold.

As shown in FIG. 8, the physiological parameter confidence measurement system 800 also has a parameter estimator 810, a physiological data reference 814 and a confidence measurer 818. The parameter estimator 810 derives one or more physiological parameter estimates, \hat{P}, 812 based upon the physiological data 810. The parameter estimate or estimates 812 are used to select one or more data clusters 816 from the physiological data reference 814. In an embodiment, the physiological data reference 814 is a collection of predetermined physiological data organized in data clusters. For example the physiological data reference 814 may contain clinically-derived physiological data organized according to corresponding values of a physiological parameter determined by a “gold standard” instrument. In a particular embodiment, the physiological data are NP ratios obtained for various physiological parameters, such as SpO₂, HbCO, HbMet, Hbt, fractional oxygen saturation, bilirubin or glucose to name a few, as measured with a standardized cooximeter, for example. In an embodiment, the physiological data reference 814 is a non-volatile memory or other data storage device containing predetermined physiological data. The confidence measurer 818 uses the physiological data 808 and the selected data cluster or data clusters 816 to generate the confidence indicator 824, the probe-off indicator 826 or both.

A confidence measurement and confidence indicator, as described herein, may be combined with other signal quality and data confidence measurements and indicators, such as those described in U.S. Pat. No. 6,996,427 titled Pulse Oximetry Data Confidence Indicator and U.S. Pat. No. 6,606,511 titled Pulse Oximetry Pulse Indicator, both patents assigned to Masimo Corporation, Irvine, CA and incorporated by reference herein. A probe off measurement and probe off indicator as described herein may be combined with other probe off measurements and indicators, such as those described in U.S. Pat. No. 6,654,624 titled Pulse Oximeter Probe-Off Detector and U.S. Pat. No. 6,771,994 titled Pulse Oximeter Probe-Off Detection System, both patents assigned to Masimo Corporation, Irvine, CA and incorporated by reference herein.

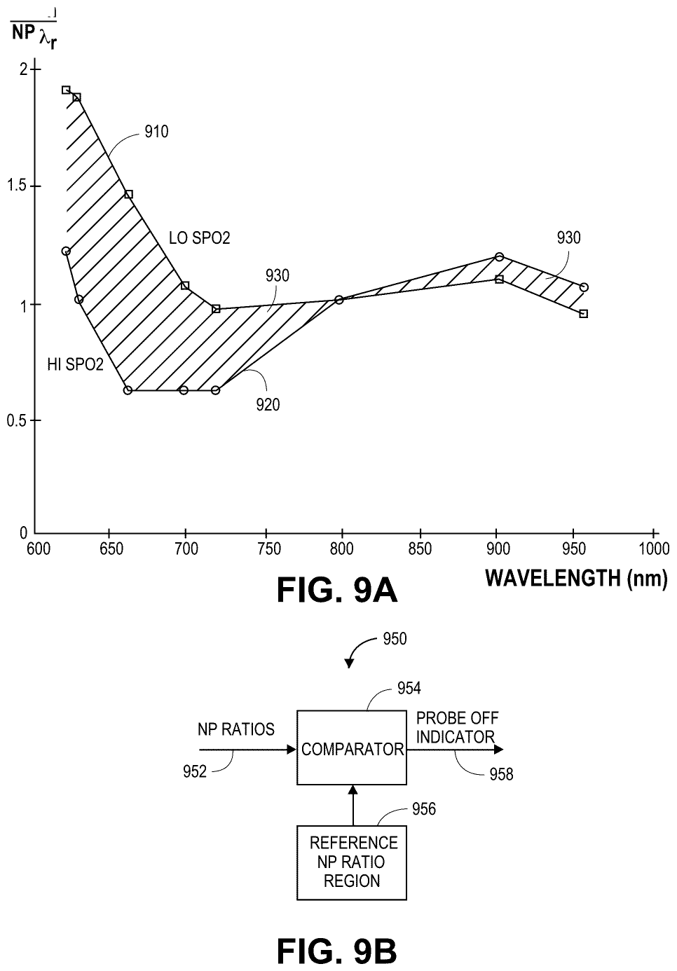

FIG. 9A illustrates NP ratio versus wavelength curves computed from a multiple wavelength sensor, such as described in the U.S. Patent Application titled “Multiple Wavelength Sensor,” referenced above. In this example, the sensor emits eight wavelengths (620, 630, 660, 700, 730, 805, 905 and 960 nm). As with FIGS. 8A and 8B, the confidence measurement derived from the embodiments shown in FIGS. 9A and 9B can be used to adjust the number of active wavelengths that is used by the physiological measurement system 300.

Shown in FIG. 9A is a low oxygen saturation curve 610, e.g. SpO₂=70% and a high oxygen saturation curve 620, e.g. SpO₂≈100%. By comparison, a conventional two wavelength pulse oximetry sensor, as described above, results in a single point on a particular curve. Advantageously, the NP ratio curves 910, 920 represent a tissue profile that can be compared to a particular sensor response to determine if a physiologically significant measurement has been made. In an embodiment, the NP ratio curves 910, 920 delineate the boundaries of a physiologically significant NP ratio region 930. Although described above with respect to SpO₂, such regions or boundaries can be derived for other physiological parameters such as HbCO, HbMet, Hbt, fractional oxygen saturation, bilirubin or glucose to name a few.

FIG. 9B illustrates one embodiment of a physiological parameter confidence measurement system 950 utilizing a NP ratio region such as described with respect to FIG. 9A, above. The confidence measurement system 950 has input NP ratios 952 measured in response to a multiple wavelength sensor, reference NP ratio region 956 that delineates physiologically significant NP ratios 930 (FIG. 9A), and a comparator 954. In one particular embodiment, the NP ratio region 956 is predetermined from clinically-derived data for one or more parameters of interest, such as SpO₂, HbCO, HbMet, Hbt, fractional oxygen saturation, bilirubin or glucose, to name a few. In another particular embodiment, the NP ratio region 956 is theoretically calculated. The comparator 954 compares the input NP ratios 952 with the NP ratio region 956 and generates a probe-off indicator 958 if any, or more than a predetermine number, of the input NP ratios 952 fall outside of an NP ratio region 956.

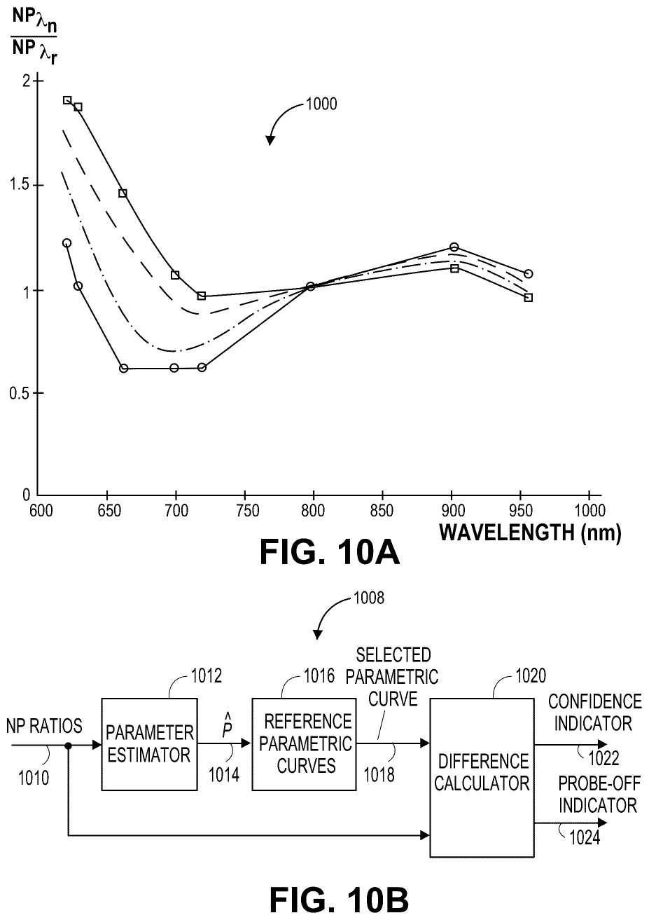

FIG. 10A illustrates a family of parametric NP ratio versus wavelength curves 1000 computed from a multiple wavelength sensor, such as referenced above. Each curve represents a different value of a measured parameter, such as SpO₂. For example, there may be a curve for each of SpO₂=70%, 75%, 80%, . . . 100%. Advantageously, such curves more precisely indicate physiologically significant multiple wavelength sensor measurements as compared to a bounded NP ratio region 930 (FIG. 9A) such as described with respect to FIGS. 9A-9B, above. The confidence measurement derived by the method shown in FIGS. 10A-10B can be used to adjust the number of active wavelengths that is used by the physiological measurement system 300.

FIG. 10B illustrates another embodiment of a physiological parameter confidence measurement system 1008 utilizing parametric NP ratio curves, such as described with respect to FIG. 10A, above. The confidence measurement system 1008 has input NP ratios 1010 measured in response to a multiple wavelength sensor, a parameter estimator 1012, reference parametric curves 1016 and a difference calculator 1020. The parameter estimator 1012 inputs the NP ratios 1010 so as to generate a parameter estimate 1014, such as SpO₂, HbCO, HbMet, Hbt, fractional oxygen saturation, bilirubin or glucose, to name a few. The estimated parameter 1014 selects one or more of the reference parametric curves 1016, which are predetermined from clinically-derived data that is stored in memory or data that is mathematically pre-calculated or calculated in real time and stored in memory. The difference calculator 1020 measures the difference between the NP ratios 1010 and the selected parametric curve 1016. For example, a mean-squared error calculation can be made between the input NP ratios 1010 and the selected parametric curve 1018. The resulting difference calculation is used as a confidence measure or translated into a confidence measure and a confidence indicator output 1022 is generated accordingly. Alternatively, or in addition to a confidence measure, a probe off condition can be indicated if the difference calculation is larger than a predetermined value or the confidence measure is less than a predetermined value. In another embodiment, a correlation calculator is used in place of the difference calculation. The confidence measurement derived from the embodiments shown in FIGS. 10A-10B can also be used to adjust the number of active wavelengths that is used by the physiological measurement system 300.

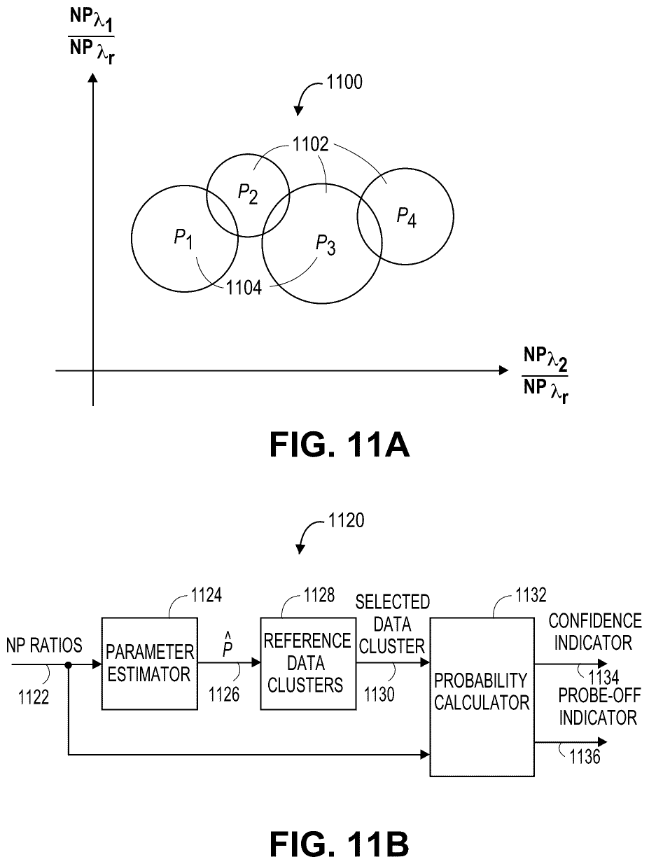

FIG. 11A illustrates a family of data clusters 1100 shown in two dimensions by way of example. Each data cluster 1100 represents NP ratios clinically measured across a population for specific values 1104 of a selected parameter P, such as P₁, P₂, P₃ and P₄ as shown. Each data cluster 1100 defines a region 1102 of NP ratios measured for a particular parameter value 1104 and has a probability distribution, such as a normal distribution, over the indicated region 1102.

For example, the clinical data can be organized as a table of known values of P, corresponding NP ratios measured over a population, and the relative number of occurrences of particular NP ratio values for each value of P. The relative number of occurrences of particular NP ratio values for a particular value of P yields an NP ratio probability distribution for that value of P. Thus, each P value 1104 in the table has a corresponding data cluster 1100 of measured NP ratios and an associated probability distribution for those NP ratios.

FIG. 11B illustrates yet another embodiment of a physiological parameter confidence measurement system 1120 utilizing NP data clusters and corresponding probability distributions, such as described with respect to FIG. 11A, above. The confidence measurement system 1120 has input NP ratios 1122 measured in response to a multiple wavelength sensor, a parameter estimator 1124, reference data clusters 1128 and a probability calculator 1132. The parameter estimator 1124 inputs the NP ratios 1122 so as to generate a parameter estimate 1126, such as described with respect to other embodiments, above. In an embodiment, the reference data clusters 1128, such as described with respect to FIG. 11A, are stored in a memory device, such as an EPROM. The estimated parameter 1130 is compared with the reference data clusters 1140 so as to determine the closest region 1102 (FIG. 11A) or closest overlapping portion of two regions 1102 (FIG. 11A). The probability calculator 1132 computes a probability based upon the distribution above the selected region 1102 (FIG. 11A). A confidence measure is also derived based upon the calculated probability. In a particular embodiment, the confidence measure is the calculated probability. A confidence indicator 1134 is generated in response to the confidence measure. In an embodiment, if the confidence probability or the calculated confidence measure is below a predetermined threshold, a probe-off indicator 1136 is generated. In particular embodiments, the confidence indicator 1134 or probe-off indicator 1136 or both may be alphanumeric or digital displays, optical indicators or alarms or similar audible indicators, to name a few. The confidence measurement derived from the embodiments shown in FIGS. 11A-11B can also be used to adjust the number of active wavelengths that is used by the physiological measurement system 300.

### Automatic Wavelength Adjustment During Calibration

Besides utilizing the confidence measurements derived from the methods and systems shown in FIGS. 8A-11B for automatic wavelength adjustment, embodiments of the physiological measurement system 300 can also automatically adjust the number of active wavelengths based on the results of a calibration process.

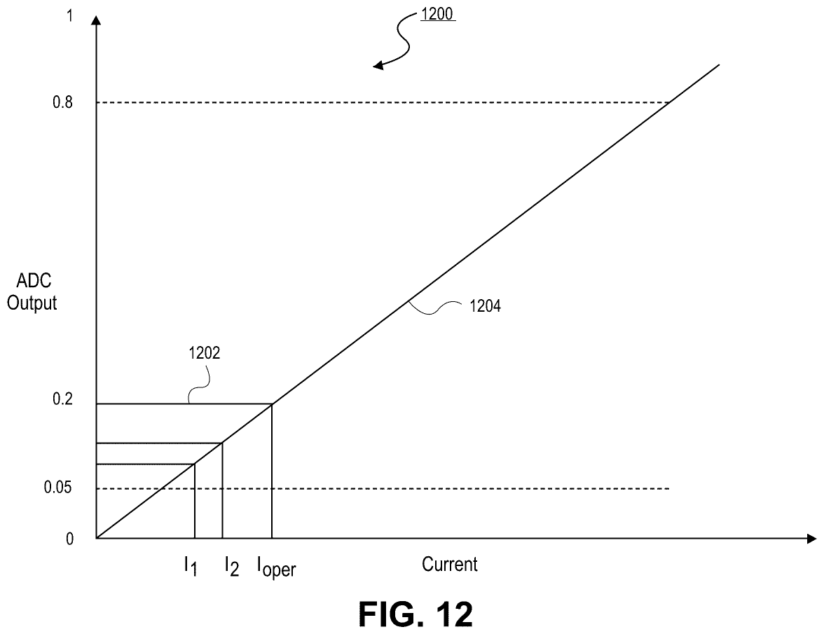

FIG. 12 shows a graph 1200 illustrating a signal calibration process performed by the physiological measurement system 300. The graph plots the Analog-to-Digital Conversion (ADC) signal output (i.e. the digitalized sensor signal) against the current supplied to an LED in the emitter assembly 332. The ADC output may be from the front-end 348 shown in FIG. 4A, for example. As shown, the ADC signal output ranges from 0 to 1 on a normalized scale. In an embodiment, an ideal range of output is preferably between 0.05 to 0.80, with an ideal operational output at about 0.2. The ideal range of output provides a proper determination of physiological data measurements.

In an embodiment, the physiological measurement system 300 performs calibration by sending a small test current through each of the LEDs that is used in emitting optical radiation at the full set of active wavelengths (e.g. the LEDs shown in TABLE 1). The system can, for example, send a 5 milliamp current, as denoted by the symbol I₁ in graph 1200. The detector then records the detected signal after tissue attenuation. A sample input-output is shown in line 1204, which illustrates the ratio of measured, digitalized sensor signal to the input current provided to the LED. The calibration can then send an additional, larger test current through the LED, e.g. 10 milliamps, as denoted by the symbol I₂. Based on the level of the measured sensor signal(s) in response to the one or more test currents provided to the LED, the physiological measurement system 300 can determine whether a sensor signal output in the acceptable range can be obtained when a larger operational current is applied. For example, the physiological measurement system 300 can use the measured outputs from the test currents to extrapolate a likely sensor signal output 1202 (shown in FIG. 12 as having a normalized ADC of 0.2) based on an anticipated operational current Iₒₚₑᵣ. In an embodiment, the DSP 346 performs these determination calculations. In other embodiments, they are performed by other components such as the front end 348.

In an embodiment, the calibration performs the same or similar test for each of the LEDs that is used in emitting optical radiation at the full set of active wavelengths, and determines whether the extrapolated signal output for the LED(s) for each individual wavelength is acceptable. In the example configuration shown in TABLE 1, where there is a one-to-one correspondence between LEDs and wavelengths, the calibration process would determine whether the extrapolated signal output for each LED is within the acceptable range. In an embodiment, the extrapolation takes into account that while each active LED may be driven by a different amount of operational current, an overall gain is applied to all active LEDs in the emitter array. Therefore, in an embodiment, the calibration process also attempts to determine an operational current for each active LED in order to have all sensor signals fall within the acceptable ADC range, as illustrated in the example in FIG. 12.

In an embodiment, if one or more extrapolated signal outputs for a particular wavelength are not in the acceptable range, the physiological measurement system 300 uses a fewer number of active wavelengths, i.e., the associated LED(s), than the full set of active wavelengths. For example, in an embodiment where the full set of active wavelengths comprises eight wavelengths, if any of the eight wavelengths returns an unacceptable result in calibration, the reduction can go from eight active wavelengths to two. The two can be of the wavelengths 660 nm (red) and 905 nm (IR), the two needed for providing a SpO2 reading. In an embodiment, the LED(s) for the two active wavelengths are activated at a longer duty cycle (½ cycle/wavelength) than when the full set of active wavelengths is used (⅛ cycle/wavelength).

In other embodiments, the number of active wavelength is first reduced from eight to four, and then from four to two. In embodiments in which the physiological measurement system includes twelve active wavelengths, the number can be progressively reduced from twelve to eight to four to two, if the calibration results necessitate such a reduction. In other embodiments, the physiological measurement system 300 does not follow a pre-set reduction routine but instead attempts to maximize the number of physiological data measurements that can be obtained given the number of wavelengths that pass the calibration test. Thus, for example, instead of reducing from twelve to two when one wavelength fails the calibration test, the physiological measurement system 300 can reduce to ten, if the remaining ten can all be used to determine physiological data measurements.

### Additional Embodiments

In an embodiment, the physiological measurement system 300 may return at least one physiological data measurement even when the detected signal does not support a full set of physiological data measurements. For example, if the detected signal indicates that a patient's perfusion is too low to support an Hb measurement but can otherwise support a SpO2 measurement, the physiological measurement system 300 may return the SpO2 measurement.

In an embodiment, the patient's perfusion level is used in the afore-mentioned confidence calculations. In an embodiment, the observed perfusion index is used as a factor in determining confidence. In another embodiment, the confidence level is determined based on perfusion alone. For example, an observed perfusion index that is outside of an acceptable range (e.g. below a threshold) would lead to a low confidence level.

In an embodiment, the physiological measurement system 300 provides user options for configuring the use of less than a full set of wavelengths. The options allow a user to configure the physiological measurement system 300 to specify the manner in which the number of wavelengths are reduced based on user-specified or pre-specified confidence level(s). For example, a user can configure the physiological measurement system 300 to use two wavelengths if the confidence level drops below a certain user-specified or pre-specified level. In another embodiment, a user can configure a confidence level below which a particular physiological data measurement is not returned by the physiological measurement system 300.

A multiple wavelength sensor with automatic wavelength adjustment has been disclosed in detail in connection with various embodiments. These embodiments are disclosed by way of examples only and are not to limit the scope of the claims that follow. One of ordinary skill in art will appreciate many variations and modifications.

1-28. (canceled)

29. An optical non-invasive physiological measurement system, the system comprising:
a plurality of light emitting sources capable of emitting light at a plurality of wavelengths, each of the plurality of the light emitting sources being configured to emit light at particular wavelength of a set of wavelengths;
a sensor capable of detecting the emitted light after attenuation by body tissue and outputting a signal that is useable for measuring at least one physiological parameter of the body tissue; and
a processor configured to:
determine, based on the signal of the sensor, a measurement of the at least one physiological parameter;
determine, based on the signal of the sensor, a confidence indicator associated with the measurement of the at least one physiological parameter; and
adjust a number of the plurality of light emitting sources that are used in operation based on the confidence indicator.

30. The system of claim 29, wherein the processor is further configured to adjust the number of the plurality of wavelengths by activating the plurality of light emitting sources configured to emit light at two wavelengths of the set of wavelengths if the confidence indicator is determined to be below a threshold.

31. The system of claim 30 wherein the threshold is 80%.

32. The system of claim 30, wherein the two wavelengths are 660 nm and 905 nm.

33. The system of claim 30, wherein the two wavelengths can be used to calculate a SpO2 reading.

34. The system of claim 29, wherein the processor is further configured to adjust by activating the plurality of light emitting sources configured to emit light at the set of wavelengths if the signal quality is determined to be sufficient to support the set of wavelengths.

35. The system of claim 34 wherein the set of wavelengths comprises eight wavelengths.

36. The system of claim 34 wherein the set of wavelengths comprises twelve wavelengths.

37. The system of claim 29, wherein the processor is further configured to adjust by activating a number of the plurality of light emitting sources that maximizes the number of physiological parameters measured if the signal quality is determined to be insufficient to support the set of wavelengths.

38. The system of claim 29, wherein the processor is configured to adjust the number of the plurality of light emitting sources that are used in operation based on a perfusion measurement made by the system.

29. The system of claim 29 wherein the perfusion measurement is based on a perfusion index.
